(19) United States
(12) Patent Application Publication
(10) Pub. No.: US 2017/0096476 A1
(43) Pub. Date: Apr. 6, 2017

(54) METHODS AND COMPOSITIONS RELATED TO AMYLOID-BETA-42 OLIGOMERS

(71) Applicant: Rakez Kayed, Galveston, TX (US)

(72) Inventor: Rakez Kayed, Galveston, TX (US)

(73) Assignee: The Board of Regents of the University of Texas System, Austin, TX (US)

(21) Appl. No.: 15/283,779

(22) Filed: Oct. 3, 2016

Related U.S. Application Data
(60) Provisional application No. 62/236,800, filed on Oct. 2, 2015.

(51) Int. Cl. C07K 16/18; G01N 33/53; C07K 14/47

(52) U.S. Cl. CPC C07K 16/18; C07K 14/4711; G01N 33/5308; C07K 2317/32; C07K 2317/20; C07K 2317/33

(57) ABSTRACT

Embodiments are directed to compositions comprising a conformation-dependent antibody that specifically binds oligomeric Aβ-42, and the methods of using the same.

### PRIORITY

The present application claims priority to U.S. Application No. 62/236,800 filed Oct. 2, 2015, which is incorporated herein by reference in its entirety.

### BACKGROUND

Embodiments of this invention are directed generally to biology, medicine, and neuroscience. In certain aspects, embodiments are directed to compositions and methods related to antibodies that bind specifically to Aβ-42 oligomers.

The presence of extracellular plaques composed of amyloid-beta (Aβ) protein and intracellular neurofibrillary tangles comprised of tau protein pathologically characterize Alzheimer's disease (AD), a progressive, irreversible neurodegenerative disorder. Aβ is a 39-42 amino acid peptide formed by the proteolytic cleavage of amyloid precursor protein (APP) by β and γ secretases. The two predominant isoforms of Aβ formed through this processing, Aβ-40 and Aβ-42, have very distinct properties even though they have high sequence similarity (Sanchez et al. (2011) Journal of the American Chemical Society 133:6505-08). Aβ-42 has a much faster aggregation rate compared to Aβ-40 due to the two unique carboxy-terminal residues of Aβ-42, isoleucine and alanine, which promote Aβ-42 polymerization (Jarrett et al. (1993) Biochemistry 32:4693-97; Esbjorner et al. (2014) Chemistry & biology 21:732-42). Furthermore, Aβ-40 primarily exists as a monomer, while Aβ-42 is in trimer/tetramer and monomer equilibrium (Chen and Glabe (2006) J Biol Chem 281:24414-22). It has also been shown that diffuse plaques are comprised primarily of Aβ-42, while mature plaques are comprised of Aβ-40, indicating that Aβ-42 is processed to Aβ-40 as plaques mature and that amyloid deposition begins with Aβ-42 (Iwatsubo et al. (1994) Neuron 13:45-53). In addition, unlike Aβ-40, Aβ-42 has been shown to promote the phosphorylation and aggregation of tau in vivo (Hu et al. (2014) Mol Neurodegener 9:52). These results support the idea that Aβ-42 is more aggregation-prone and the more pathogenic species compared to Aβ-40.

Furthermore, insoluble Aβ fibrils constituting classic plaques were thought to be responsible for the neurodegenerative changes associated with AD for many years (Selkoe (2001) Physiological reviews 81, 741-766). However, growing evidence implicates soluble oligomers as the more toxic species, and the extent of oligomer formation and assembly correlates better with disease progression and cognitive dysfunction (Kayed et al. (2010) Mol Neurodegener 5:57; Tomic et al. (2009) Neurobiol Dis 35:352-58). Aβ oligomers have been shown to bind membrane receptors, form pores in cell membranes, and form intracellular aggregates that lead to pathological events including mitochondrial dysfunction and proteasome impairment (Kayed and Lasagna-Reeves (2013) J Alzheimers Dis 33(Suppl 1):567-78). These Aβ oligomers are able to induce other aggregation-prone proteins, including α-syn, PrP, and TDP-43, to assume oligomeric conformations. These proteins can then seed tau aggregation, resulting in neurodegeneration (Guerrero-Munoz et al. (2014) Neurobiol Dis 71:14-23).

There remains a need for the development of agents that specifically bind amyloid-beta peptide aggregates or oligomers.

### SUMMARY

Considering the propensity of Aβ-42 aggregation and the increasing evidence pointing to the toxicity of Aβ oligomers, the inventor developed a conformation-dependent antibody, called VIA, against oligomeric Aβ-42. VIA is reactive to Aβ-42 aggregates and does not detect Aβ-40 oligomers, Aβ monomer, or APP. The transgenic APP_{K670L,M671N} Tg2576 mouse is one of the most extensively studied AD models and is characterized by the over-production and deposition of Aβ protein (Hsiao et al. (1996) Science 274:99-102). Similar to other amyloid precursor protein (APP) models, the Tg2576 exhibits hyperphosphorylated tau, but it does not develop neurofibrillary tangles (NFTs) (Sturchler-Pierrat et al. (1997) PNAS USA 94:13287-92; Maia et al. (2013) Science translational medicine 5:194re192). The inventor has established the specificity of the novel VIA antibody to Aβ-42 oligomers in vitro and ex vivo in human AD and Tg2576 mice.

Amyloid-beta (Aβ) oligomers have emerged as the most toxic species in Alzheimer's disease (AD) and other amyloid pathologies. Also, Aβ-42 peptide is more aggregation-prone compared to other Aβ isoforms. The inventor designed and synthesized a small peptide of repeated sequence containing the last three amino acids, Val-40, Ile-41 and Ala-42 (VIA) of Aβ-42 that was subsequently aggregated and used to generate a novel antibody, VIA. As described below, human AD and Tg2576 mouse brain samples were examined using VIA in combination with other amyloid-specific antibodies, confirming the specificity of VIA to oligomeric Aβ-42. Moreover, it was found that VIA does not recognize classic amyloid plaques composed of fibrillar Aβ or Aβ-40 ex vivo. Since VIA recognizes a distinct epitope specific to Aβ-42 oligomers, it can have broad use for examining the accumulation of these oligomers in AD and other neurodegenerative diseases. VIA can also be used in immunotherapy to prevent neurodegenerative effects associated with Aβ-42 oligomers.

Certain embodiments are directed to antibody compositions comprising an antibody or antibody fragment that specifically binds an aggregate comprising a peptide having the amino acid sequence of VIAVIA (SEQ ID NO:1), wherein the antibody or antibody fragment does not bind Aβ-40, Aβ-42 monomer, or amyloid precursor protein (APP). The antibody or antibody fragment can be a rabbit antibody or rabbit antibody fragment. The composition can be in the form of a pharmaceutically acceptable formulation. The antibody composition can be administered to a subject in need of such composition. A subject can be at risk of developing an Aβ-42 related disease, e.g., Alzheimer's disease, or can be suspected of having an Aβ-42 related disease or condition.

Certain aspects are directed to methods of detecting Aβ-42 oligomers comprising the step of contacting a sample with an Aβ-42 oligomer specific antibody that specifically binds an aggregate comprising a peptide having the amino acid sequence of VIAVIA (SEQ ID NO:1) and detecting the binding of the antibody to an amyloid oligomer in the sample. The sample can be a patient sample. The patient sample can comprise plasma, cerebrospinal fluid (CSF), brain tissue, neuronal tissue, or muscle tissue. In certain aspects the detecting step is by immunoassay. In a further aspect the Aβ-42 oligomer specific antibody is coupled to a detectable agent and/or a support. The detectable agent is a radioactive marker, a nucleic acid, a fluorescent label, or an enzymatic label. The support can be glass, polymer, paper, or plastic support.

Other embodiments are directed to an antigen comprising an aggregate of a peptide having the amino acid sequence of SEQ ID NO:1. In certain aspects the antigen is comprised in an immunogenic formulation.

Certain embodiments are directed to a method of preparing an Aβ-42 oligomer specific antibody, comprising the steps of administering a peptide aggregate of a peptide having the amino acid sequence of VIAVIA (SEQ ID NO:1) to a mammal and isolating antibodies or cells that produce antibodies that specifically bind to Aβ-42 oligomers and do not bind Aβ-42 monomers, Aβ-40, or amyloid precursor protein (APP). Certain aspects are directed to an Aβ-42 oligomer specific antibody composition produced by the methods described herein.

The term “antibody”, as used herein, broadly refers to any immunoglobulin (Ig) molecule or amyloid oligomer binding peptide derived from an antibody including any functional fragment, mutant, variant, or derivation thereof, which retains the essential epitope binding or Aβ-42 oligomer binding features of an Ig molecule that specifically binds the Aβ-42 oligomer. In certain aspects, an antibody is a monoclonal antibody or a single chain antibody. In still further aspects, the antibody is a recombinant antibody segment that retains Aβ-42 oligomer specific binding. Typically, antibody fragments compete with the intact antibody from which they were derived for specific binding to an antigen. Fragments include separate heavy chains, light chains Fab, Fab′ F(ab′)2, Fabc, and Fv. Fragments are produced by recombinant DNA techniques, or by enzymatic or chemical separation of intact immunoglobulins.

An “isolated antibody”, as used herein, is intended to refer to an antibody that is substantially free of other antibodies having different antigenic specificities.

As used herein, the term “humanized antibody” is an antibody or a variant, derivative, analog or segment thereof that immunospecifically binds to an antigen of interest and that comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, 85%, 90%, 95%, 98% or 99% identical to the amino acid sequence of a non-human antibody CDR. A humanized antibody comprises substantially all of at least one, and typically two, variable domains (Fab, Fab′, F(ab′)2, FabC, Fv) in which all or substantially all of the CDR regions correspond to those of a non-human immunoglobulin (i.e., donor antibody) and all or substantially all of the framework regions are those of a human immunoglobulin consensus sequence. Preferably, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain.

As used herein, the term “antigen” is a molecule or complex capable of being bound by an antibody or T-cell receptor. An antigen is additionally capable of inducing a humoral immune response and/or cellular immune response leading to the production of B- and/or T-lymphocytes. B-lymphocytes respond to foreign antigenic determinants via antibody production, whereas T-lymphocytes mediate cellular immunity. The structural aspect of an antigen, e.g., three dimensional conformation or modification (e.g., phosphorylation), which gives rise to a biological response is referred to herein as an “antigenic determinant” or “epitope.” Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and usually at least 5, 6, 7, 8, 9, or 10 amino acids in a unique spatial conformation. In certain aspects, a peptide having an amino acid sequence of VIAVIA (SEQ ID NO:1) is used as an antigen, in particular aggregates comprising a plurality of peptides having an amino acid sequence of SEQ ID NO:1.

As used herein the phrase “immune response” or its equivalent “immunological response” refers to a humoral (antibody mediated), cellular (mediated by antigen-specific T cells or their secretion products) or both humoral and cellular response directed against a protein, peptide, or polypeptide of the invention in a subject or a donor subject. A donor subject is one in which an antibody is generated and isolated, the isolated antibody is then administered to a second subject. Treatment or therapy can be an active immune response induced by administration of an immunogen or a passive therapy affected by administration of an antibody, antibody-containing material, or primed T-cells.

The phrase that an antibody or molecule “specifically binds” or is “specifically immunoreactive” to a target refers to a binding reaction that is determinative of the presence of the target or a target containing a binding determinant in the presence of a heterogeneous population of other molecules or sample components. Thus, under designated immunoassay conditions, a specified molecule binds preferentially to a particular target and does not bind in a significant amount to other biologics or moieties present in the sample. Specific binding of an antibody to a target under such conditions requires the antibody be selected for its specificity to the target. A variety of immunoassay formats may be used to select antibodies specifically immunoreactive with a particular protein. For example, solid-phase ELISA immunoassays are routinely used to select monoclonal antibodies specifically immunoreactive with a protein. See, e.g., Harlow and Lane (1988), for a description of immunoassay formats and conditions that can be used to determine specific immunoreactivity. Specific binding between two entities means an affinity of at least 10⁶, 10⁷, 10⁸, 10⁹ M⁻¹, or 10¹⁰ M⁻¹. Affinities greater than 10⁸M⁻¹ are preferred.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B. Specificity of anti-Aβ-42 oligomeric VIA antibody. Dot blot using VIA and 6E10 against varying concentrations of prepared recombinant Aβ-42 and Aβ-40 oligomers indicates that VIA is specific to Aβ-42 oligomers and does not recognize Aβ-40 (FIG. 1A). Western blot using VIA and 6E10 against Aβ-40 and Aβ-42 confirms VIA specificity and indicates that VIA does not recognize monomeric Aβ-42 (FIG. 1B).

FIGS. 2A-2D. Toxicity assay of SH-SY5Y cells treated with preparations of Aβ-42. Images of cells treated with 2 μM of Aβ-42 fibrils, Aβ-42 oligomers, and Aβ-42 oligomers with VIA (FIGS. 2A, 2B, and 2C respectively). Percentage of cells affected with each sample is shown in D. Aβ-42 oligomers were more toxic to cells compared to Aβ-42 fibrils and Aβ-42 oligomers alone were more toxic than oligomers blocked with VIA. Bars and error bars represent means and standard deviations, respectively (*p<0.05, **p<0.01). Scale bar=50 μm.

FIGS. 3A-3O. VIA recognizes intracellular perinuclear aggregates of Aβ-42 and does not recognize amyloid plaques in human AD brains. Single-labeling, bright-field images with anti-oligomeric Aβ-42 antibody VIA in control (FIG. 3A), frontal cortex, and hippocampus of AD brains (FIGS. 3B and 3C respectively). Epifluorescence double immunofluorescence images (FIGS. 3D-3O) of AD frontal cortex and control brain sections labeled with VIA (FIGS. 3E, 3H, 3K, and 3N), 6E10 (FIGS. 3D and 3G), and anti-Aβ-42 antibody (FIGS. 3J and 3M). Some colocalization was observed between 6E10 and VIA in AD brains and VIA did not recognize classic amyloid plaques (FIG. 3I). Significant colocalization was observed between VIA and anti-Aβ-42 antibody in AD brains (FIG. 3O). Minimal immunoreactivity was detected with VIA antibody (FIGS. 3E and 3K) in control brain samples. Scale bar=50 μm.

FIG. 4A-4L. VIA recognizes both prefibrillar and fibrillar Aβ-42 oligomers in human AD brains. Epifluorescence double immunofluorescence images (FIGS. 4A-3I) and confocal images (FIGS. 4J-4L) of AD frontal cortex and control brain sections labeled with VIA (FIGS. 4B, 4E, 4H, and 4K), A11 (FIGS. 4A and 4D), and OC (FIGS. 4G and 4J). Significant colocalization was observed between A11 and VIA in AD brains. Colocalization was observed between OC and VIA in micro-deposits in AD brains (FIG. 4I) however, VIA did not recognize OC-positive amyloid plaques (FIGS. 4I and 4L). Significant minimal immunoreactivity was detected with VIA antibody (FIG. 4B) in control brain samples. Scale bar=50 μm (C, F, and I). Scale bar=10 μm (L).

FIGS. 5A-5L. VIA recognizes intracellular aggregates of Aβ-42 in activated astrocytes and microglia in the frontal cortex of human AD brains. Epifluorescence microscopy images of AD and control brain sections labeled with anti-oligomeric Aβ-42 antibody VIA (FIGS. 5B, 5E, 5H, and 5K), GFAP (FIGS. 5A and 5D), and Iba1 (FIGS. 5G and 5J). Significant colocalization was observed between VIA and GFAP (FIG. 5F) and Iba1 (FIG. 5L) in AD brains. Minimal immunoreactivity was detected with VIA antibody in age-matched control brain samples (FIGS. 5B and 5H). Scale bar=50 μm.

FIGS. 6A-6G. VIA recognizes Aβ-42 oligomers but not amyloid plaques in coronal sections of Tg2576 mice. Aβ-42 aggregates were seen in sections of 6, 10, and 14-month-old Tg2576 mice (FIGS. 6B, 6C, and 6D respectively) stained with VIA and 4G8. VIA-negative amyloid plaques were observed at 14 months (FIG. 6D). No VIA immunoreactivity was detected in control C57BL/6 mice (FIG. 6A). Confocal microscopy images of 14 month Tg2576 brain sections labeled with Aβ-40 (FIG. 6E) and VIA (FIG. 6F). No colocalization was observed between Aβ-40 and VIA (FIG. 6G). Scale bar=50 μm (FIGS. 6A-6D). Scale=10 μm.

FIGS. 7A-7J. VIA recognizes Aβ-42 oligomers in the hippocampus of 10 and 14-month-old Tg2576 mice. Bright-field images emphasizing the CA1 region of the hippocampus labeled with VIA antibody in control wild-type C57BL/6 (FIG. 7A), 10-month-old Tg2576 (FIG. 7B) and 14-month-old Tg2576 mice (FIG. 7C). Epifluorescence microscopy double fluorescence images of control wild-type C57BL/6 (FIGS. 7D, 7H), 10 month Tg2576 (FIGS. 7E, 7I) and 14 month Tg2576 mice (FIGS. 7F-7G, 7J) stained with VIA (FIGS. 7D-7J) and 4G8 (FIG. 7D-7G) or OC (FIGS. 7H-7J). Note image FIG. 7G is a less magnified view (20× image) of the same brain section shown in FIG. 7F and shows a VIA-negative plaque at the end of the CA1 region. VIA does not recognize inert plaques observed in 14-month-old mice (FIGS. 7G, 7J). Scale bar=50 μm.

FIG. 8. Schematic representation of antigen design used to generate an A0-42 oligomer-specific antibody. The amino acid sequence VIA represent the last three amino acids of A0-42. Thus, a repeated sequence of six amino acids, VIAVIA was synthesized and subsequently aggregated to produce a highly specific immunogenic antigen. This aggregated VIAVIA antigen was used to produce antibody specific for aggregated A0-42.

FIG. 9. Aβ-42 oligomers co-localize with α-synuclein in human AD brains forming perinuclear co-aggregates. Control brain sections were negative for co-localization between Aβ-42 oligomers and α-synuclein. Aβ-42 oligomers also co-localize with tau oligomers in AD brains suggesting the presence of hybrid oligomers. Scale bar=50 μm.

FIG. 10. Positive or negative indicates the presence or absence respectively of co-localization between A0-42 oligomers and other proteins in AD or control cases. Aβ-42 oligomers were shown to form co-aggregates with TDP-43, PrP, and insulin in human AD cases by double immunofluorescence analysis. IP analysis indicated the presence of amyloid complexes comprised of oligomeric proteins in AD brain homogenates.

FIG. 11. Model of secondary amyloidosis in Alzheimer's Disease. A0-42 oligomers cross-seed the formation of other protein oligomers, resulting in the formation of mixed and complex pathologies in AD.

### DESCRIPTION

Amyloid peptides and proteins adopt various distinct assembly states that differ in their degree of pathogenicity. Conformation-dependent antibodies that are specific for different assembly states can provide insight into their unique characteristics and significance in disease. The inventor has developed a novel conformation-dependent antibody called VIA that successfully recognizes recombinant preparations of Aβ-42 oligomers and is specific to Aβ-42 oligomers in human AD brains and Tg2576 mice. Furthermore, VIA does not recognize inert amyloid plaques and is not reactive to Aβ-40 in human AD brains and Tg2576 mice. Since VIA recognizes a distinct epitope specific to toxic Aβ-42 oligomers, it can have broad applications for detecting and studying the accumulation of Aβ-42 oligomers in AD and other neurodegenerative diseases.

Amyloid oligomers have been previously classified into two distinct categories based on their unique conformations. Prefibrillar or Type 1 amyloid oligomers are A11-reactive and have been suggested to spread throughout different regions of the brain causing neural dysfunction and leading to cognitive deficits (Kayed et al. (2007) Mol Neurodegener 2:18; Liu et al. (2015) Cell reports 11:1760-71). Fibrillar or Type 2 oligomers are OC-reactive and have a parallel β-sheet structure similar to amyloid plaques (Kayed et al. (2007) Mol Neurodegener 2:18; Liu et al. (2015) Cell reports 11:1760-71). It has been suggested that fibrillary oligomers seed the formation of amyloid plaques and that these plaques subsequently are involved in the formation of micro-deposits of fibrillar oligomers (Liu et al. (2015) Cell reports 11:1760-71). These oligomers are confined in the vicinity of amyloid plaques and are thus considered less toxic compared to prefibrillar oligomers, which have been shown to spread. The results are consistent with these previous findings. VIA and OC staining in mice showed significant deposits of Aβ-42 fibrillar oligomers in the vicinity of amyloid plaques in human AD and Tg2576 mice. Furthermore, it was shown that VIA is specific to Aβ-42 species of both oligomer classes, and thus may be used in conjunction with various antibodies to further study these oligomeric species.

Also, neuroinflammation is often associated with AD pathology contributed by microglial cells and astrocytes in the brain. Activated microglia and accumulation of astrocytes are found surrounding Aβ deposits (Tuppo and Arias (2005) The international journal of biochemistry & cell biology 37:289-305). VIA-positive Aβ-42 oligomers were found in activated astrocytes and microglia, indicating that these oligomers may play a role in inflammation in AD. Astrocytes most likely take up Aβ oligomers, which are later formed into Aβ annular protofibrils (APFs). APFs cause harmful metabolic changes within astrocytes and thus compromise neuronal health (Lasagna-Reeves and Kayed (2011) FEBS Lett 585:3052-57). Moreover, previous studies have shown that Aβ oligomers stimulate primary microglia and that activated human microglia increase the expression of Aβ-42 in neuroblastoma cells (Lee et al. (2015) Neurobiol Aging 36:42-52; Sondag et al. (2009) J Neuroinflammation 6:1). The current observations and these previous results suggest that Aβ-42 oligomers activate astrocytes and microglia, which release inflammatory markers that increase the formation of Aβ-42 oligomers, promoting neurodegeneration. Injured neurons further activate astrocytes and microglia resulting in a feed-forward cycle of inflammation and cell death contributing to AD (Zhao et al. (2011) J Neuroinflammation 8:150).

The results suggest that Aβ-42 oligomers are a viable therapeutic target for AD. Aβ is considered to be critical since it lies upstream of tau pathology (Clinton et al. (2010) J Neurosci 30:7281-89). Furthermore, it has been well established that aggregated Aβ induces tau phosphorylation, mislocalization, and aggregation in animal models and cell cultures (De Felice et al. (2008) Neurobiol Aging 29:1334-47; Gotz et al. (2001) Science 293:1491-95; Busciglio et al. (1995) Neuron 14:879-88; Ferrari et al. (2003) J Biol Chem 278:40162-68; Ittner et al. (2010) Cell 142:387-97). In addition, Aβ oligomers seed tau oligomerization, an important event in AD pathogenesis since tau oligomers cause mitochondrial and synaptic dysfunction (Lasagna-Reeves et al. (2010) Biochemistry 49:10039-41; Lasagna-Reeves et al. (2011) Mol Neurodegener 6:39).

The results described herein indicate that the novel VIA antibody can be used successfully in passive immunotherapy to target Aβ-42 oligomers. The same aggregated antigen can be used to generate Aβ-42 oligomer-specific mouse monoclonal antibodies. VIA was shown to be specific to aggregated Aβ-42 in human AD brains and Tg2576 mice. Since VIA does not recognize classic amyloid plaques, a passive immunotherapy approach using VIA can prevent plaque disruption and potential side effects, including inflammation (Gilman et al. (2005) Neurology 64:1553-62). Furthermore, since VIA is specific to Aβ-42 and does not recognize Aβ-40, which is associated with vascular amyloid, a VIA-based immunotherapy approach would lower the risk hemorrhagic stroke (Herzig et al. (2006) Brain pathology 16:40-54). Although previous Aβ active and passive immunotherapy approaches for the treatment of AD have shown mixed results in clinical trials, it is believe that targeting a highly specific aggregation or assembly state of AP, in this case, Aβ-42 oligomers, rather than all forms of AP, may be a more beneficial approach (Patel (2015) Managed care 24:19; Panza et al. (2014) Expert review of clinical immunology 10: 405-19).

Embodiments of the invention include compositions and methods for producing and using antibodies that bind specifically to Aβ-42 oligomers—in particular antibodies that specifically bind to aggregates of peptides having the amino acid sequence of VIAVIA (SEQ ID NO:1). Embodiments relate to novel antibodies and compositions that can be employed in the treatment, detection, or evaluation of a group of disorders and abnormalities associated with amyloid protein, such as Alzheimer's disease. Certain aspects further relate to pharmaceutical compositions comprising these antibodies or peptides, and to the use of these compositions for the preparation of medicaments for the treatment of diseases or conditions associated with amyloid proteins.

Certain embodiments are related to peptides, oligomers, antibodies, and antibody fragments for use in various embodiments of the present invention. For example, antibodies generated to a peptide or aggregates comprising a peptide having an amino acid sequence of SEQ ID NO:1 are identified for specific binding to Aβ-42 oligomers.

### I. ANTI-Aβ-42 OLIGOMER ANTIBODIES

Certain embodiments of the invention are directed to antibodies that specifically bind aggregates comprising a peptide having the amino acid sequence of VIAVIA (SEQ ID NO:1) and specifically bind Aβ-42 oligomers.

To generate antibodies an immunostimulatory amount of inoculum is administered to a mammal and the inoculated mammal is then maintained for a time sufficient for the antigenic composition to induce protective antibodies. The antibodies can be isolated to the extent desired by well-known techniques such as affinity chromatography (Harlow and Lane, 1988). Antibodies can include antiserum preparations from a variety of commonly used animals e.g., goats, primates, donkeys, swine, horses, guinea pigs, rats, or man. The animals are bled and serum recovered.

Inocula for antibody production are typically prepared by dispersing the antigenic composition (e.g., a peptide aggregate of a peptide having an amino acid sequence of SEQ ID NO:1) in a physiologically tolerable diluent such as saline or other adjuvants suitable for human use to form an aqueous composition.

Antibodies to the antigen are subsequently collected from the sera of the host. The antibody can be affinity purified against the antigen. An antigen composition of the present invention can be administered to a recipient who then acts as a source of antibodies, produced in response to challenge an antigen composition comprising an aggregate of a peptide having the amino acid sequence of SEQ ID NO:1. A subject thus treated would donate plasma from which antibody would be obtained via conventional plasma fractionation methodology; or would donate antibody producing cells that could be cultured and used for production of antibodies in culture. The isolated antibody would be administered to the same or different subject in order to impart resistance against or treat an Aβ-42 related disease or condition.

One method of producing monoclonal antibodies includes hyperimmunization of an appropriate donor, generally a mouse, with the antigen. Isolation of antibody producing cells, typically spleen cells, is then carried out. These cells are fused to a cell characterized by immortality, such as a myeloma cell, to provide a fused cell hybrid (hybridoma) which can be maintained in culture and which secretes the appropriate monoclonal antibody. The cells are then cultured, in bulk, and the monoclonal antibodies harvested from the culture media for use. By definition, monoclonal antibodies are specific to a single epitope (e.g., VIAVIA peptide aggregates). Monoclonal antibodies often have lower affinity constants than polyclonal antibodies raised against similar antigens for this reason.

Monoclonal antibodies may also be produced ex vivo by use of primary cultures of splenic cells or cell lines derived from spleen (Anavi, 1998). In order to produce recombinant antibody messenger RNAs from antibody producing B-lymphocytes of animals, or hybridoma are reverse-transcribed to obtain complementary DNAs (cDNAs). Antibody cDNA, which can be full length or partial length, is amplified and cloned into a phage or a plasmid. The cDNA can be a partial length of heavy and light chain cDNA, separated or connected by a linker. The antibody, or antibody fragment, is expressed using a suitable expression system to obtain recombinant antibody. Antibody cDNA can also be obtained by screening pertinent expression libraries. Alternatively, monoclonal Fv fragments can be obtained by screening a suitable phage display library. Monoclonal antibodies may be humanized or part humanized by known methods.

Optionally, an antibody or preferably an immunological portion of an antibody, can be chemically conjugated to, or expressed as, a fusion protein with other proteins. For purposes of this specification and the accompanying claims, all such fused proteins are included in the definition of antibodies or an immunological portion of an antibody.

Typically, antibodies are comprised of four polypeptide chains, two heavy (H) chains and two light (L) chains. In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FRI, CDR1, FR2, CDR2, FR3, CDR3, FR4. Immunoglobulin molecules can be of any type (e.g., IgG, IgE, IgM, IgD, IgA and IgY), class (e.g., IgG1, IgG2, IgG3, IgG4, IgA1 and IgA2) or subclass.

The framework and CDR regions of an antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In a preferred embodiment, such mutations, however, will not be extensive. Usually, at least 80%, preferably at least 85%, more preferably at least 90%, and most preferably at least 95% of the humanized antibody residues will correspond to those of the parental FR and CDR sequences.

Certain embodiments are directed to anti-Aβ-42 antibodies that specifically bind AP-42 aggregates and are conformation-dependent, do not bind native precursor or monomer, and/or do not bind Aβ-40 or APP. In certain aspects, the antibodies are generated by vaccinating mice or other animal host with aggregated of the VIAVIA peptide or peptide aggregate.

In certain embodiments, all or part of the peptides or proteins of the invention can be synthesized in solution or on a solid support in accordance with conventional techniques. Various automatic synthesizers are commercially available and can be used in accordance with known protocols. See, for example, Stewart and Young, (1984); Tam et al., (1983); Merrifield, (1986); and Barany and Merrifield (1979). Alternatively, recombinant DNA technology may be employed wherein a nucleotide sequence that encodes a peptide or polypeptide of the invention is inserted into an expression vector, transformed or transfected into an appropriate host cell and cultivated under conditions suitable for expression.

In a certain aspects, an immunogenic composition according to the invention comprises an aggregate of the VIAVIA peptide.

### II. THERAPEUTIC AND DIAGNOSTIC METHODS

Certain methods include treatment for or diagnosis of an amyloid related disease or condition caused by amyloid oligomers. Furthermore, in some examples, treatment comprises administration of other agents commonly used to treat amyloid related disease. Therapeutic and diagnostic aspects of the invention include antibodies that specifically bind to Aβ-42 aggregates via conformation specific epitope(s). The present invention provides for amyloid disease therapeutics that can induce a specific immune response against Aβ-42 oligomers or provide passive immunity to Aβ-42 oligomers.

One use of the immunogenic compositions of the invention is to prophylactically treat a subject in early stages of an amyloid related disease by inoculating a subject, particularly once a risk of developing an amyloid related disease has been indicated. In certain aspects, a “risk” means symptoms being presented or having a familial history of an amyloid related disease, i.e., a genetic predisposition.

Such prophylactic therapy can be imparting a passive immunity to the subject. Passive immunity may be imparted to a patient or subject by administering to the patient immunoglobulins (Ig) or fragments thereof and/or other immune factors obtained from a donor or other non-patient source having a known immunoreactivity. In other aspects recombinantly produced antibodies can also be used to confer passive immunity. In other aspects, an antigenic composition of the present invention can be administered to a subject who then acts as a source or donor for globulin, produced in response to challenge from the composition (“hyperimmune globulin”), that contains antibodies directed against Aβ-42 oligomers. A subject thus treated would donate plasma from which hyperimmune globulin would then be obtained, e.g., via conventional plasma-fractionation methodology, and administered to another subject in order to impart resistance against or to treat an amyloid related disease. See U.S. Pat. Nos. 6,936,258; 6,770,278; 6,756,361; 5,548,066; 5,512,282; 4,338,298; and 4,748,018, each of which is incorporated herein by reference in its entirety, for exemplary methods and compositions related to passive immunity.

The compositions and related methods of the present invention, particularly administration of an immunogenic composition comprising a peptide aggregate comprising a peptide having an amino acid sequence of SEQ ID NO:1 or an antibody that specifically binds an aggregate of SEQ ID NO:1, may also be used in combination with the administration of traditional therapies.

In one aspect, it is contemplated that a traditional therapy is used in conjunction with a composition comprising an aggregate comprising a peptide having an amino acid sequence of SEQ ID NO:1 or an Aβ-42 oligomer specific antibody treatment. Alternatively, the therapy may precede or follow the traditional therapy by intervals ranging from minutes to weeks. In embodiments where the other agents and/or a proteins or polynucleotides are administered separately, one would generally ensure that a significant period of time did not expire between the time of each delivery, such that the therapeutic composition would still be able to exert an advantageously combined effect on the subject. In such instances, it is contemplated that one may administer both modalities within about 12-24 h of each other and, more preferably, within about 6-12 h of each other. In some situations, it may be desirable to extend the time period for administration significantly; however, where several days (2, 3, 4, 5, 6 or 7) to several weeks (1, 2, 3, 4, 5, 6, 7 or 8) lapse between the respective administrations.

Various combinations of therapy may be employed, for example immunogenic compositions described herein or Aβ-42 oligomer specific antibody therapy as a first therapeutic agent and a traditional amyloid related disease therapy as a second therapeutic agent. The two or more therapeutic agents can be co-formulated, or they can be separately formulated and co-administered simultaneously or consecutively in any order. Therapeutics that restore the deficit (defect), or malfunctioning, in the chemical messengers of the nerve cells (neurotransmitters), in particular the cholinesterase inhibitors (ChEIs) such as tacrine and rivastigmine, have been shown to improve symptoms of amyloid related diseases. ChEIs impede the enzymatic degradation of neurotransmitters thereby increasing the amount of chemical messengers available to transmit the nerve signals in the brain.

In certain aspects, anti-Aβ-42 antibodies of the invention can be used to detect the effects of small molecules on the persistence or diminution of oligomers in high-through put screening assays.

Compositions of the invention can be used to characterize oligomeric species in human brain, serum, CSF, and transgenic animals.

Certain aspects are directed to the use of the antigen in a vaccine specifically targeting Aβ-42 oligomers. Furthermore, the anti-Aβ-42 antibodies can be provided as a passive immunotherapy, intrabodies, humanized mAb agents for the detection and/or treatment of amyloid related diseases. In particular aspects, the anti-Aβ-42 antibodies can be used in producing diagnostic kits.

The antibodies described herein can be used in immunohistochemical and biochemical methods in combination with other well characterized antibodies for qualitative and quantitative analysis of Aβ-42 levels, localization, and posttranslational modifications of oligomers in brain samples and CSF samples using direct ELISA, immunoprecipitation/western, and sandwich ELISA.

Anti-Aβ-42 antibodies can be used in transgenic animal models for assessment of therapeutic efficacy. For example, amyloid oligomers can be studied in brain from the AD models Tg 2576 and APP/PS1 mice, as well as the P301L amyloid (JNPL3).

### III. ADMINISTRATION AND FORMULATION

As discussed above, the compositions can be administered to a subject having, suspected of having, or at risk of developing an Aβ-42 related disease. Therapeutic compositions are administered in a manner compatible with the dosage formulation, and in such amount as will be therapeutically effective. The quantity to be administered depends on the subject to be treated. Precise amounts of active ingredient to be administered depend on the judgment of the practitioner. Suitable regimes for initial administration and boosters are also variable, but are typified by an initial administration followed by subsequent administrations.

The manner of application may be varied widely. Any of the conventional methods for administration of a polypeptide as a therapeutic are applicable. In certain aspects an Aβ-42 specific antibody that specifically binds an aggregate comprising a peptide having an amino acid sequence of SEQ ID NO:1 can be administered into the cerebrospinal fluid of the brain or spine. In certain embodiments, an immunogenic composition of the invention may be inhaled (e.g., U.S. Pat. No. 6,651,655, which is specifically incorporated by reference). Such compositions would normally be administered as pharmaceutically acceptable compositions that include physiologically acceptable carriers, buffers, or other excipients.

In certain instances, it will be desirable to have multiple administrations of the composition, e.g., 2, 3, 4, 5, 6 or more administrations. The administrations can be at 1, 2, 3, 4, 5, 6, 7, 8 to 5, 6, 7, 8, 9, 10, 11, 12 day or week intervals, including all ranges there between.

Administration of the antibody or immunogenic compositions of the present invention to a patient/subject will follow general protocols for the administration of such compositions, taking into account the toxicity, if any, of the composition. It is expected that the treatment cycles would be repeated as necessary. It is also contemplated that various standard therapies, such as hydration, may be applied in combination with the described therapy.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, antibacterial and antifungal agents, isotonic and absorption delaying agents, and the like.

The proteinaceous compositions may be formulated into a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like.

A pharmaceutical composition comprising antibodies that specifically bind an aggregate comprising a peptide having an amino acid sequence of SEQ ID NO:1 and/or Aβ-42 oligomers and a pharmaceutically acceptable carrier is a further aspect of the invention that can be used in the manufacture of a medicament for the treatment or prevention of an amyloid related disease or condition.

An additional aspect of the invention is a pharmaceutical composition comprising one of more antibodies or monoclonal antibodies (or fragments thereof; preferably human or humanized) generated by using an aggregate of a peptide having an amino acid sequence of SEQ ID NO:1. It is contemplated that in compositions of the invention, there is about 0.001, 0.01, 0.1, 1, 5, μg or mg to about 0.01, 0.1, 1, 5, 10 μg or mg of total polypeptide, peptide, and/or protein per ml. The concentration of protein in a composition can be about, at least about or at most about 0.001, 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0 mg/ml, including all values and ranges there between. In certain aspects the dose range is 0.01 to 500 mg/kg, 10 to 300 mg/kg, or 0.01 to 10 mg/kg.

An effective amount of therapeutic or prophylactic composition is determined based on the intended goal, i.e., treatment or amelioration of an amyloid related disease. The term “unit dose” or “dosage” refers to physically discrete units suitable for use in a subject, each unit containing a predetermined quantity of the composition calculated to produce the desired responses discussed above in association with its administration, i.e., the appropriate route and regimen. The quantity to be administered, both according to number of treatments and unit dose, depends on the protection desired. Precise amounts of the composition also depend on the judgment of the practitioner and are peculiar to each individual. Factors affecting dose include physical and clinical state of the subject, route of administration, intended goal of treatment (alleviation of symptoms versus cure), and potency, stability, and toxicity of the particular composition.

### IV. EXAMPLES

The following examples as well as the figures are included to demonstrate preferred embodiments of the invention. It should be appreciated by those of skill in the art that the techniques disclosed in the examples or figures represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

A. Results

In-Vitro Specificity of VIA to Aβ-42 Oligomers.

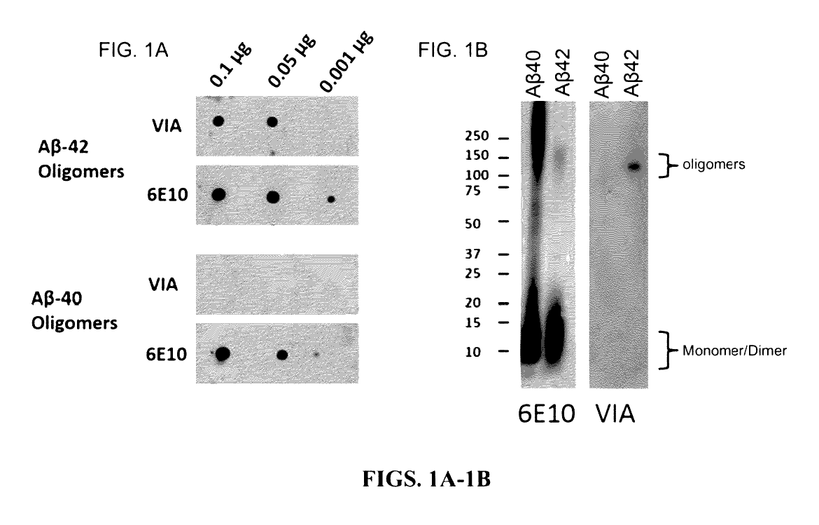

Specificity of VIA to Aβ-42 oligomers was determined by dot blot and Western blot analysis using prepared recombinant Aβ samples. Dot blot analysis using various amounts of Aβ oligomers indicated that VIA is specific for AP-42 oligomers (FIG. 1A). Signal was obtained using 0.1 and 0.05 μg of oligomeric Aβ-42 protein sample, although no signal was detected using 0.001 μg of sample or same concentrations of AP-40 oligomers. As expected, 6E10 recognized both Aβ-40 and 42. Western blot analysis verified VIA specificity to Aβ-42 and indicated that VIA does not recognize monomeric Aβ-42, which has a weight of approximately 4.5 kDa. VIA recognized SDS-stable conformational variants of Aβ-42 of high molecular weight between 100-150 kDa (FIG. 1B).

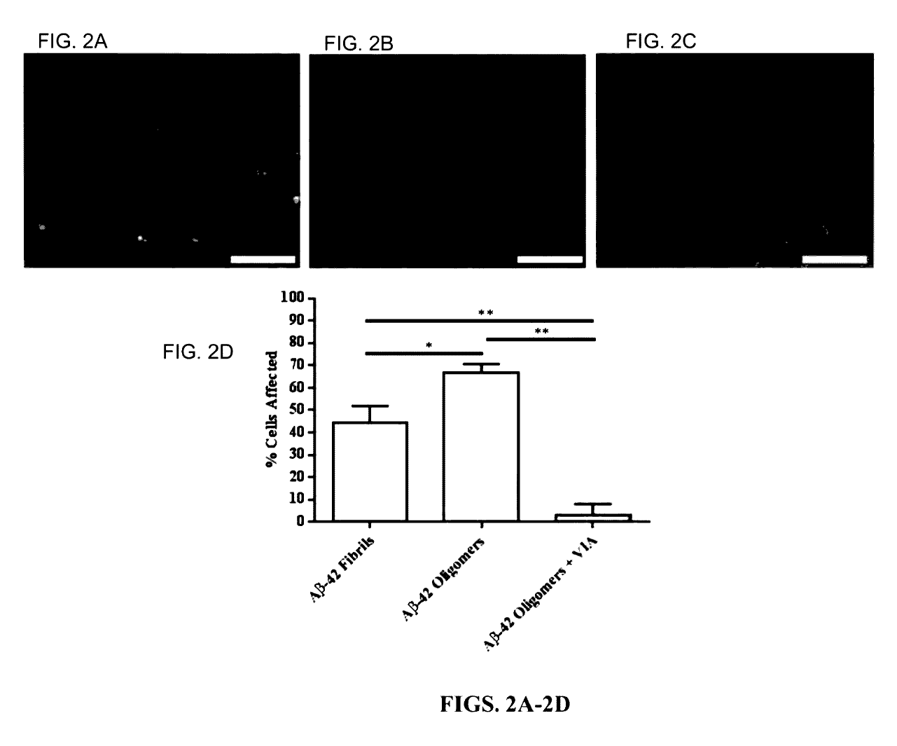

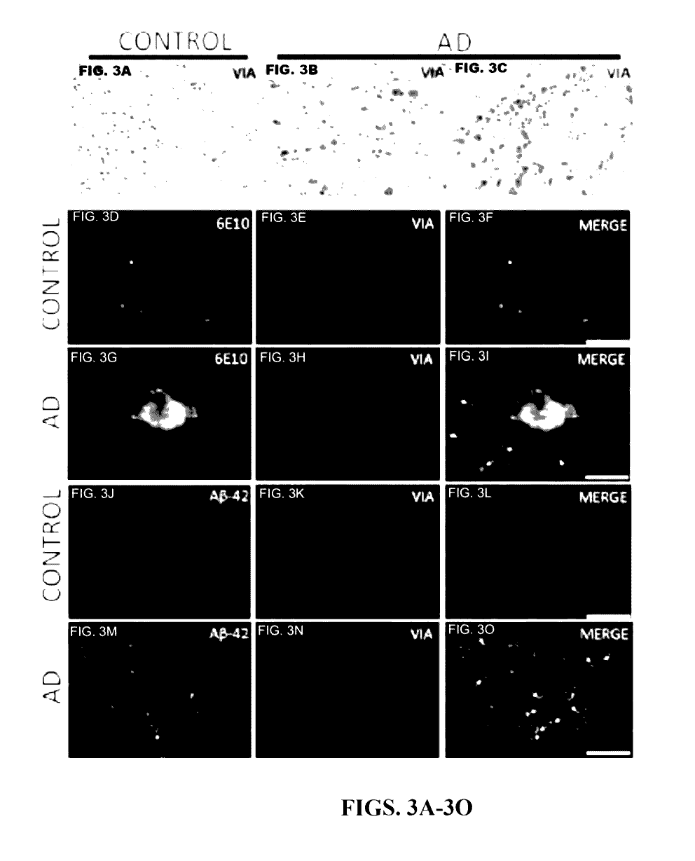

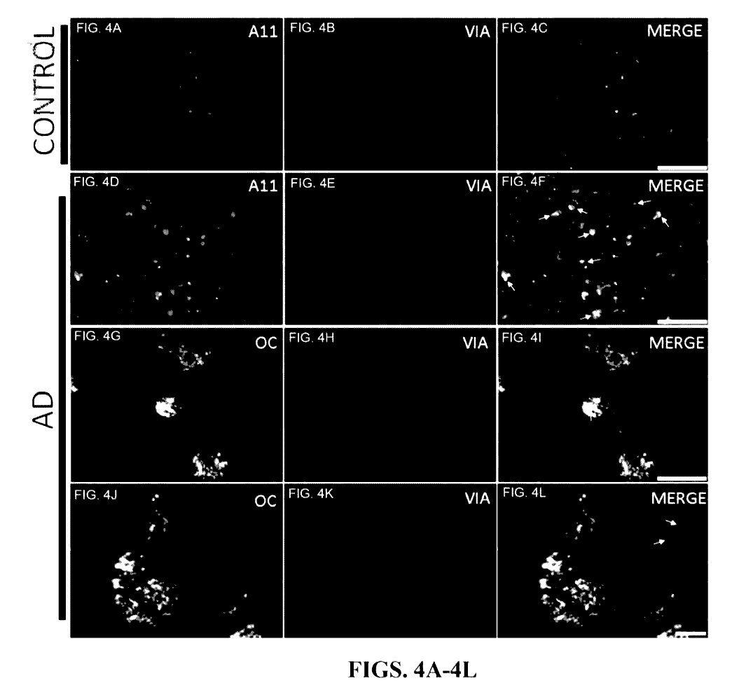

In addition, SH-SY5Y cells were treated with Aβ-42 oligomers, Aβ-42 fibrils, and Aβ-42 oligomers incubated with VIA antibody to examine the toxicity of these preparations. Aβ-42 oligomers were much more toxic to cells with 66.9% of cells affected after treatment compared to Aβ-42 fibrils, with only 44.4% of cells affected (FIGS. 2A, 2B and 2D). Moreover, Aβ-42 oligomers and VIA antibody complex induced less cell toxicity with 2.9% of cells affected, indicating that VIA binds to Aβ-42 oligomers and neutralizes their toxic effects (FIGS. 2C and 2D).

Ex Vivo Specificity of VIA to Aβ-42 Oligomers.

Immunohistochemistry and double immunofluorescence labeling using VIA in combination with other amyloid antibodies were used to determine VIA specificity in human AD brain samples. Immunohistochemical analysis with VIA staining revealed significant perinuclear aggregates of Aβ-42 in the frontal cortex and hippocampus of these brain samples (FIG. 3B-3C). Staining with VIA and amyloid-specific 6E10 antibodies indicated that VIA does not recognize classic amyloid plaques (FIG. 3I). Furthermore, VIA-positive oligomers showed some colocalization with 6E10 in certain regions of the frontal cortex, suggesting that the 6E10 epitope is exposed in these oligomers (FIG. 4I). Also, significant colocalization was observed between VIA and anti-Aβ-42 antibody, confirming VIA specificity to aggregates of Aβ-42 rather than Aβ-40 (FIG. 3O).

Studies have suggested that at least two different types of amyloid oligomers with distinct conformations exist: prefibrillar or Type 1 oligomers that are reactive to A11 antibody, and fibrillar or Type 2 oligomers that are reactive to OC antibody which specifically identifies amyloid fibrils (Kayed et al. (2007) Mol Neurodegener 2:18; Glabe (2008) J Biol Chem 283:29639-43; Liu et al. (2015) Cell reports 11:1760-71). Fibrillar oligomers have a parallel β-sheet structure similar to that present in amyloid plaques while prefibrillar oligomers have a unique structure distinct from that of fibrillar oligomers (Kayed et al. (2007) Mol Neurodegener 2:18; Glabe (2008) J Biol Chem 283:29639-43). To determine VIA immunoreactivity to these different classes of Aβ oligomers, control and human AD brains were stained with VIA and A11 or OC. Significant colocalization was observed between VIA and A11 in the frontal cortex of human AD brains, while minimal VIA and A11-positive signal was observed in age-matched controls, suggesting that VIA recognizes Aβ-42 prefibrillar aggregates in these diseased brains (FIGS. 4C and 4F). Double staining with VIA and OC antibodies indicated that OC-positive amyloid plaques are not recognized by VIA (FIGS. 4I and 4L). However, colocalization was observed between VIA and OC in micro-deposits comprised of fibrillar oligomers that are OC-positive, indicating that VIA recognizes Aβ-42 fibrillar oligomers. Thus, VIA recognizes both prefibrillar and fibrillar Aβ-42 oligomers.

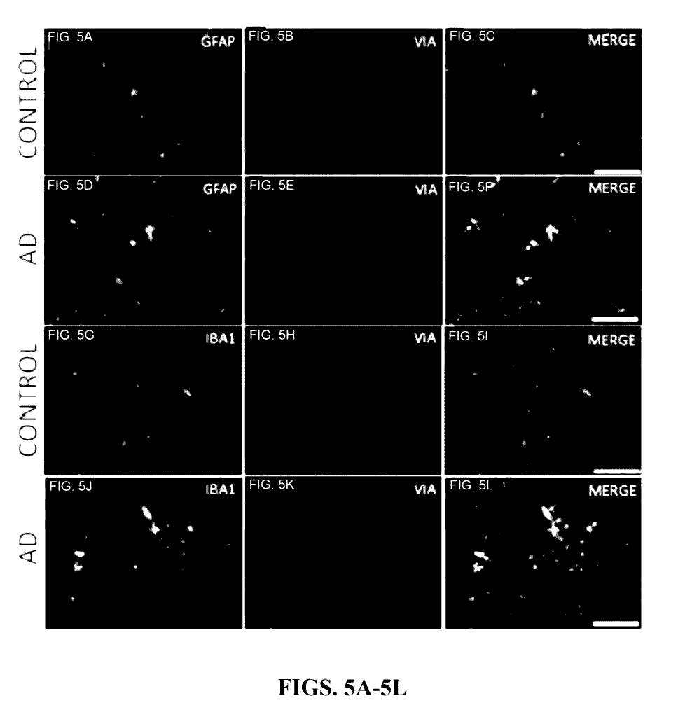

In addition, AD brains and age-matched control brains were stained with VIA and two neuroinflammmatory markers, GFAP, a marker for activated astrocytes and Iba1, a marker for activated microglia. Colocalization was found between VIA and GFAP (FIG. 5F) and Iba1 (FIG. 5L) in AD brains. This observation is in accordance with previous studies suggesting the role of Aβ oligomers in microglia and astrocyte activated inflammation. Therefore, the results indicate that VIA recognizes pathologically toxic Aβ oligomers that are associated with inflammation in AD brains.

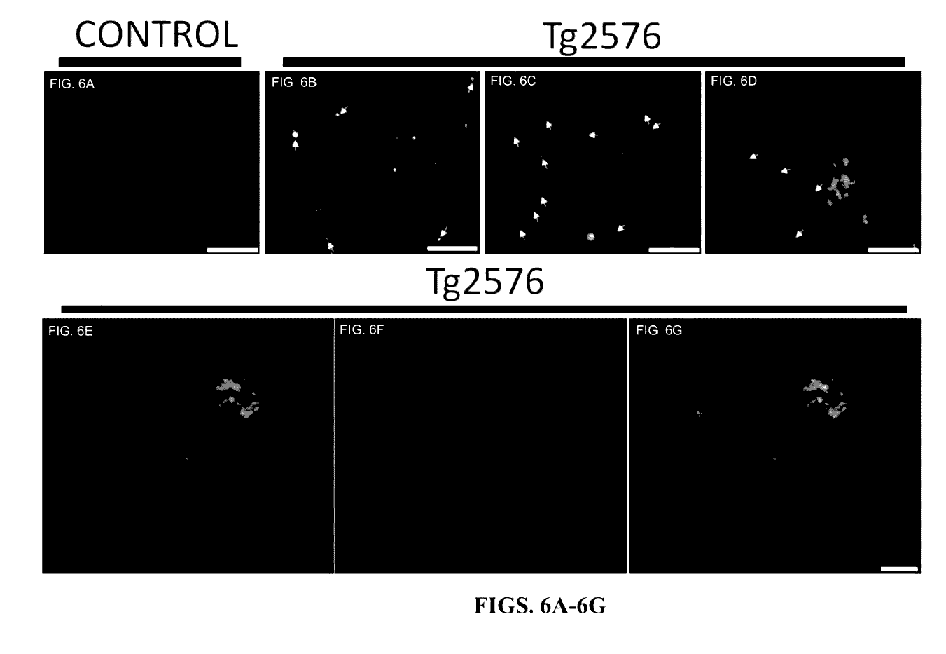

To determine VIA specificity to Aβ-42 oligomers in Tg2576 mice, coronal sections of 6, 10, and 14-month-old Tg2576 mice were stained with VIA and amyloid-specific 4G8. Microdeposits of Aβ-42 oligomers were found in the coronal sections of 6, 10, and 14-month-old Tg2576 mice (FIGS. 6B and 6D) but were not observed in C57BL/6 mice (FIG. 6A). Significant colocalization was observed between VIA and 4G8 in Tg2576 mice, suggesting the 4G8 epitope is exposed in these oligomers. Furthermore, classic amyloid plaques observed in 14-month-old Tg2576 mice were negative for VIA, confirming that VIA does not recognize Aβ fibrils, although VIA-positive oligomeric Aβ-42 deposits were found in the vicinity of the plaques (FIG. 6D). Furthermore, to confirm VIA specificity to Aβ-42 in Tg2576 mice, brain sections of 14-month-old Tg2576 mice were stained with VIA and an anti-Aβ-40 antibody. VIA showed no colocalization with Aβ-40 and did not recognize Aβ-40 plaques (FIG. 6G).

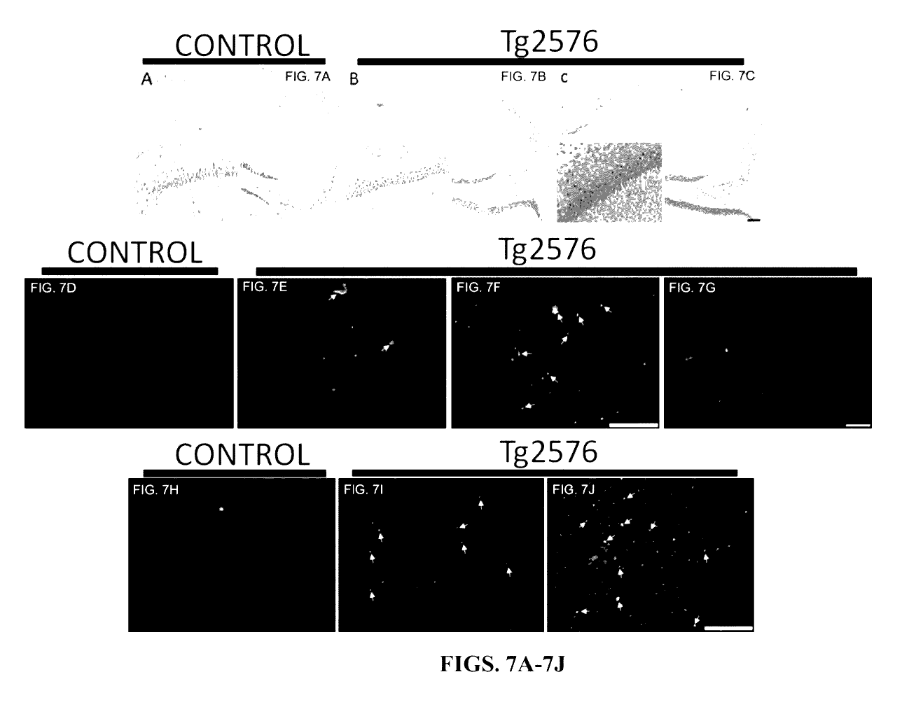

To examine the localization of Aβ-42 oligomers specifically in the hippocampus of Tg2576 mice, the hippocampi of 10 and 14-month-old Tg2576 and control 12-month-old wildtype C57BL/6 mice were stained with VIA. Significant VIA immunoreactivity was observed in the CA1 region of the hippocampus of Tg2576 mice (FIGS. 7B and 7C) while no VIA immunoreactivity was detected in the CA1 region of C57BL/6 mice (FIG. 7A). Furthermore, the hippocampi of 10 and 14-month-old Tg2576 mice were stained with VIA and 4G8 or OC. Micro-deposits of Aβ-42 oligomers positive for VIA and 4G8 were found in the CA1 region of the hippocampus in Tg2576 mice (FIGS. 7E and 7F) while 4G8-positive amyloid plaques seen in the hippocampus were VIA-negative (FIG. 7G). Also, micro-deposits positive for both VIA and OC were observed in the hippocampus of Tg2576 mice (FIGS. 7I and 7J). These VIA and OC-positive deposits were found in the vicinity of OC-positive amyloid plaques in 14-month-old mice, although the plaques were not recognized by VIA (FIG. 7J). The VIA and OC-positive deposits are most likely comprised of fibrillar oligomers since these oligomers are OC-positive (Kayed et al. (2007) Mol Neurodegener 2:18). Thus, the results suggest that VIA is able to recognize Aβ-42 fibrillar oligomers in Tg2576 mice.

Aβ-42 Oligomers Form Co-Aggregates with Aggregation-Prone Proteins in Human AD.

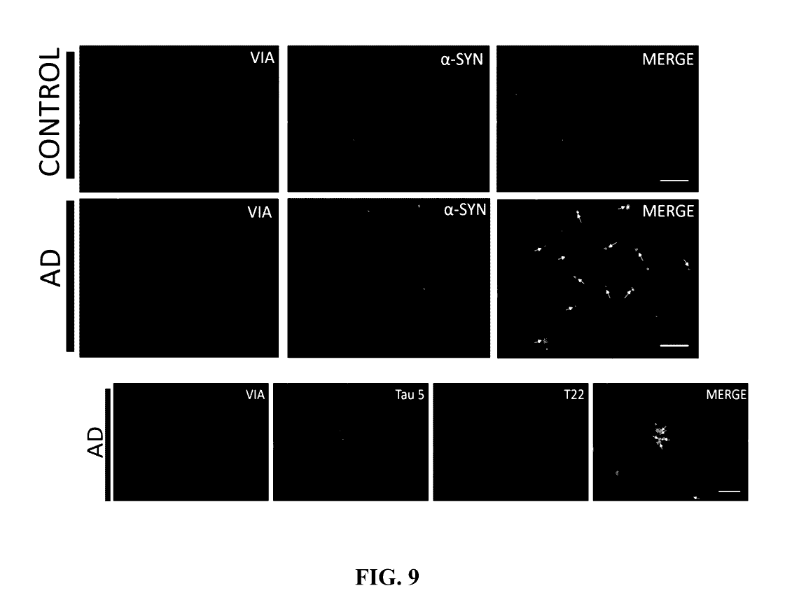

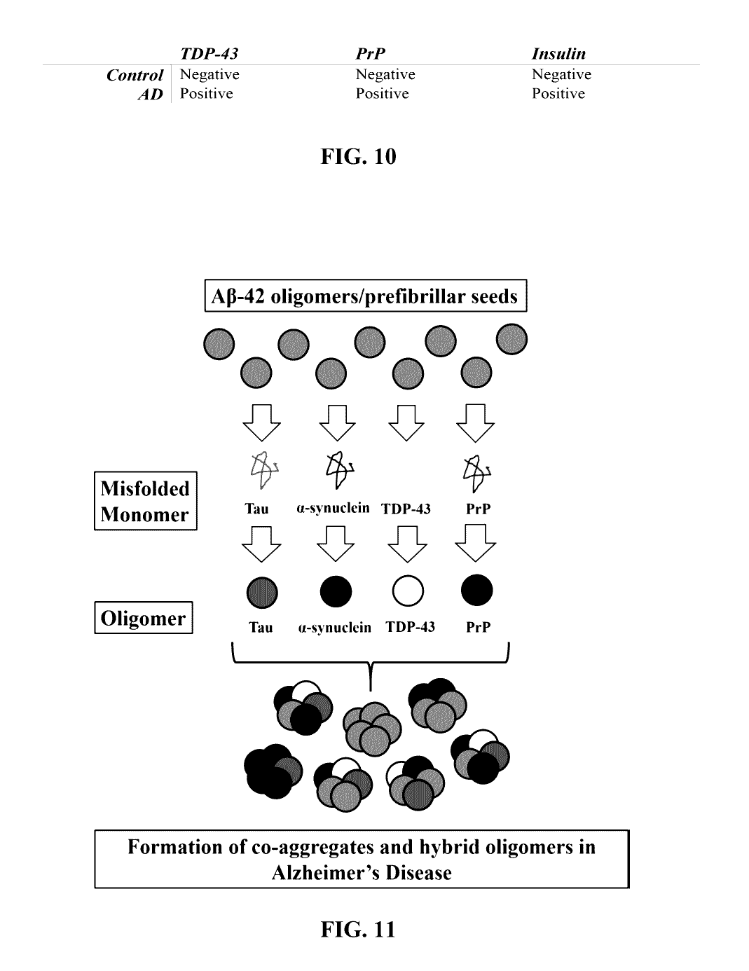

Aβ-42 oligomers were shown to co-localize with other aggregation-prone proteins in AD, including tau, α-synuclein, TDP-43, and PrP. FIG. 9 and FIG. 10 show the results of studies that indicate Aβ-42 oligomers co-localize with α-synuclein in human AD brains forming perinuclear co-aggregates. Control brain sections were negative for co-localization between Aβ-42 oligomers and α-synuclein. Aβ-42 oligomers also co-localize with tau oligomers in AD brains suggesting the presence of hybrid oligomers (FIG. 9). Studies suggest that the interaction between Aβ-42 oligomers and other proteins in AD can be explained by the cross-seeding capability of these oligomers (FIG. 11). Aβ-42 oligomers also form aggregates with insulin, which could be reducing the binding affinity of insulin for its receptor, suggesting a link between Type-2 Diabetes and AD (FIG. 10).

B. Methods

Oligomer Preparation.

Lyophilized peptides of Aβ-40 and Aβ-42 were resuspended in 50% acetonitrile/water mixture and relyophilized. Oligomers were prepared by dissolving 0.3 mg of peptide in 250 μL of hexafluoroisopropanol (HFIP) and incubating for 10-20 minutes at room temperature as described previously (Kayed et al. (2003) Science 300:486-89). Then 200 μL of the resulting solution was added to 700 μL of double-distilled H₂O (dd H₂O) in a siliconized Eppendorf tube. The samples were then stirred at 500 rpm using a Teflon-coated microstir bar for 24-48 hours at room temperature (RT). Tube caps contained holes to allow slow evaporation of HFIP.

Generation of Antigen and Oligomer-Specific VIA Antibody.

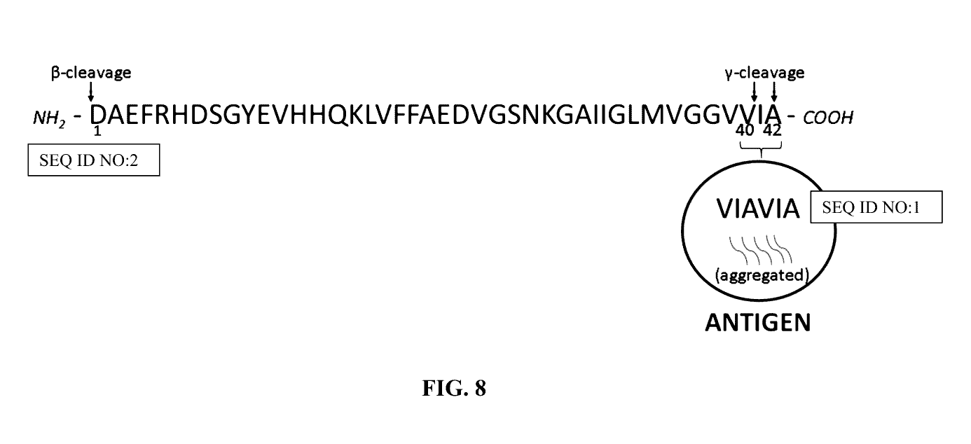

The amino acid sequence VIA (valine, isoleucine, and alanine) represents the last three amino acids of Aβ-42. Aggregated Aβ-40 and Aβ-42 share similar β-sheet structural architecture. The last two amino acids, Ile-41 and Ala-42 in Aβ-42 peptide are exposed to the solvent (Torok et al. (2002) J Biol Chem 277:40810-15), thus representing a potential binding site for antibodies targeted to its aggregated form. Although the β-sheet exhibits parallel in-register organization, some structural studies have shown that is has a tendency to shift forming different polymorphisms (Paravastu et al. (2008) PNAS USA 105:18349-54; Petkova et al. (2005) Science 307:262-65). To account for this potential shift, the antigen was extended from Ala-42 and Ile-41 to Val-40. To obtain maximum immunogenicity and appropriate peptide length for aggregation, repeated sequence VIAVIA (FIG. 8) was used. This small peptide was synthesized, subsequently aggregated and used as the antigen to generate antibody by immunizing New Zealand white rabbits (Pacific Immunology Corp. Ramona, Calif.). Antibody was purified from the sera obtained from these immunized animals using affinity chromatography.

Immunoblotting.

Protein samples (recombinant amyloid oligomers) were electrophoresed on NuPAGE 4-12% Bis-Tris gels (Invitrogen). Samples were transferred onto nitrocellulose membranes, which were subsequently blocked overnight at 4° C. using 10% non-fat dry milk in 1×TBST buffer and then washed once for 5 minutes with 1× TBST buffer. Membranes were incubated with VIA serum or purified polyclonal antibody (1:50) and 6E10 antibody (1:1000, Covance) in 5% milk-TBST for 1 hour at RT. Following washing, the membranes were then incubated with corresponding anti-rabbit or anti-mouse secondary antibodies conjugated with horseradish peroxidase (1:3000, GE Healthcare) at RT for 1 hour. Membranes were washed several times and finally developed with Super Signal West Pico chemiluminescence reagents (Thermo Scientific). For dot blots, 0.3-1.2 μL of sample was spotted, blocked, and immunolabeled as described above for Western blotting.

Toxicity Assay.

SH-SY5Y neuroblastoma cells were maintained in DMEM with 10 mM HEPES, 10% fetal bovine serum, 4 mM glutamine, penicillin (200 U/mL) and streptomycin (200 μg/mL) in 5% CO₂ at 37° C. The medium was replaced every 2 days. Cells were differentiated in serum-free DMEM with N₂ supplement and 1×10⁻⁵ M all-trans retinoic acid before use. Cells were plated at 10,000 cells/well in 96-well plates and grown overnight. The medium was removed and 2 μM of Aβ-42 oligomers, fibrils, and oligomers incubated with VIA antibody (2:1 ratio of antibody to oligomers) were added in 80 μL fresh medium. After incubation for 90 minutes at 37° C., cells were subjected to MTT colorimetric assay using Cell Proliferation I (MTT) assay kits (Roche) according to the manufacturer's directions. Each data point was determined in triplicate.

Human and Mouse Brain Selection.

Frozen brain tissue from AD patients and age-matched controls was obtained from the University of California at Irvine Alzheimer's Disease Research Center (UCI-ADRC). Sections of frontal cortex samples from moderate-to-severe Braak stage AD cases and non-demented elderly controls were used in this study.

Sections that included the hippocampus from Tg2576 mice at 6, 10, and 14 months of age that were bred at University of Texas Medical Branch (UTMB) were compared with 12-month-old C57BL/6 wild-type mice (stock #1638M; Taconic).

Immunohistochemistry.

For single-labeling, bright-field immunohistochemistry, all frozen sections were fixed in chilled methanol and treated with 3% hydrogen peroxide in 1×PBS to quench endogenous peroxidase. Sections were subsequently washed twice for 5 minutes each with 1×PBS and then blocked with 5% goat serum in PBS for 60 minutes at RT. Sections were incubated with VIA serum (1:150) overnight at 4° C. Next day, sections were washed three times with 1×PBS and were incubated with goat anti-rabbit biotinylated anti-IgG and then with avidin biotin complex (ABC) (Vectastain ABC kit, Vector Laboratories). This was followed by hematoxylin staining for nuclei.

Paraffin sections were first deparaffinized and rehydrated using xylene, 100% ethanol, 95% ethanol, 80% ethanol and distilled water. The sections were then treated for retrieving antigenic sites. Subsequent hydrogen peroxide treatment, blocking, and antibody labeling were conducted as described above.

Double Immunofluorescence.

A series of immunofluorescence experiments were conducted using VIA antibody in combination with various antibodies. Frozen brain tissue was fixed in chilled methanol followed by blocking with 5% goat serum in 1×PBS for 1 hour at RT. Sections were incubated with VIA serum (1:150) overnight at 4° C. After washing three times with 1× PBS, sections were incubated with a secondary goat anti-rabbit IgG antibody (Alexa-Fluor 568 [1:350]) for 1 hour at RT for visualization. Sections were washed three times for 10 minutes each and subsequently incubated with a second primary antibody overnight at 4° C. The amyloid-specific antibodies used were 6E10 (1:300, Covance) which recognizes amino acids 1-16 of AP, 4G8 (1:200, Covance) which is specific to 17-24 of Aβ, OC (1:300) which is specific for fibrillar Aβ, A11 (1:500) which recognizes prefibrillar oligomers, anti Aβ-42 (1:200, Abcam), and anti Aβ-40 (1:500, Covance). Anti-GFAP (1:200) and anti-Iba1 (1:100) were also used to detect activated astrocytes or microglia respectively. After washing three times, sections were incubated with the appropriate species-specific secondary antibody for 1 hour at RT. Sections were washed three times to remove unbound antibodies. Nuclei were stained with DAPI in 1×PBS (4′,6′-diamidino-2-phenylindole; [1:3000], Molecular Probes) and subsequently washed with 1×PBS and mounted (Fluoromount G, Southern Biotech).

Paraffin sections were first deparaffinized and rehydrated using xylene, 100% ethanol, 95% ethanol, 80% ethanol and distilled water. The sections were treated for antigen retrieval followed by washing. Subsequent blocking and antibody labeling were conducted as described.

List of Antibodies Used:

| Antibody | Antigen |
|---|---|
| VIA | Aβ-42 oligomers |
| 6E10 | Pan Aβ |
| T22 | Tau oligomers |
| Tau 5 | Pan Tau |
| Anti-α-synuclein | α-synuclein |
| Anti-TDP-43 | TDP-43 |
| Anti-PrP | PrP |
| Anti-Insulin | Insulin |

Image Collection.

Images were collected using an epifluorescence microscope (Nikon Eclipse 800) equipped with a CoolSnap-FX monochrome CCD camera (Photometrics) using standard Nikon fluorescein isothiocyanate (FITC), Texas Red, and DAPI filters. Image acquisition and analysis were performed using cellSens software (Olympus Life Science).

Images were also collected using a Zeiss LSM-510 Meta confocal microscope with a 63×1.20 numerical aperture water immersion objective (UTMB Optical Microscopy Core). Images were sequentially obtained using three different excitation lines (364, 488, and 543). After excitation with 364-, 488-, and 543-lasers, line emissions were collected with 385-470 nm, 505-530 nm, and 560-615 nm filters respectively. An 8-frame-Kallman-averaging with a pixel time of 2.51 μs and a pixel size of 1.60 nm were used to collect images.

Immunoprecipitation Analysis.

Soluble fractions of cortical brain homogenates from AD and control subjects were immunoprecipitated with VIA antibody and the resulting protein complexes characterized by dot blot analysis.

Statistical Analysis.

Statistical analysis on the data was performed by Excel (Microsoft). Significant p value was set to <0.05. Bars and error bars represent means and standard deviations, respectively.

1. An antibody composition comprising an antibody or antibody fragment that specifically binds an aggregate comprising a plurality of peptides having an amino acid sequence of VIAVIA (SEQ ID NO:1), wherein the antibody or antibody fragment does not bind Aβ-40, Aβ-42 monomer, or amyloid precursor protein (APP).

2. The composition of claim 1, wherein the antibody or antibody fragment is a rabbit antibody or rabbit antibody fragment.

3. The composition of claim 1, wherein the composition is a pharmaceutically acceptable formulation.

4. A method of detecting Aβ-42 oligomers comprising the step of contacting a sample with an Aβ-42 oligomer specific antibody that specifically binds an aggregate comprising a peptide having the amino acid sequence of VIAVIA (SEQ ID NO:1) and detecting the binding of the antibody to an amyloid oligomer in the sample.

5. The method of claim 4, wherein the sample is a patient sample.

6. The method claim 5, wherein the patient sample comprises plasma, cerebrospinal fluid (CSF), brain tissue, neuronal tissue, or muscle tissue.

7. The method of claim 4, wherein detecting antibody binding is by immunoassay.

8. The method of claim 7, wherein the Aβ-42 oligomer specific antibody is coupled to a detectable agent.

9. The method of claim 8, wherein the detectable agent is a radioactive marker, a nucleic acid, a fluorescent label, or an enzymatic label.

10. An antigen comprising an aggregate of a plurality of peptides having an amino acid sequence of SEQ ID NO:1.

11. The antigen of claim 10, wherein the antigen is comprised in an immunogenic formulation.

12. A method of preparing an Aβ-42 oligomer specific antibody, comprising the steps of administering a peptide aggregate of a peptide having the amino acid sequence of VIAVIA (SEQ ID NO:1) to a mammal and isolating antibodies or cells that produce antibodies that specifically bind to Aβ-42 oligomers and do not bind Aβ-42 monomers, Aβ-40, or amyloid precursor protein (APP).

13. An Aβ-42 oligomer specific antibody composition produced by the method of claim 12.


SEQUENCE LISTING (2 sequences)

SEQ ID NO: 1 - protein, 6 aa, Artificial Sequence
  Synthetic peptide
VIAVIA

SEQ ID NO: 2 - protein, 42 aa, Homo sapiens [sequence omitted]